(19) United States
(12) Patent Application Publication
(10) Pub. No.: US 2015/0056304 A1
(43) Pub. Date: Feb. 26, 2015

(54) GEL, IN PARTICULAR FOR USE IN A WOUND TREATMENT AGENT

(71) Applicant: Hans-Georg Mathé, Basel (CH)

(72) Inventor: Hans-Georg Mathé, Basel (CH)

(21) Appl. No.: 14/131,634

(22) PCT Filed: Jul. 6, 2012
(86) PCT No.: PCT/EP2012/002849

§ 371 Date: Feb. 27, 2014

(30) Foreign Application Priority Data
Jul. 8, 2011 (EP) 11005618.1

(51) Int. Cl. A61L 26/00

(52) U.S. Cl. CPC A61L 26/0004; A61L 26/0066; A61L 2300/106; A61L 2300/404
USPC 424/663

(57) ABSTRACT

A gel, in particular for use in a wound treatment agent includes an inorganic silicate as thickener; and an electrochemically activated aqueous common salt solution having a content of free chlorine of more than 200 mg/l and a conductivity of not more than 12 mS/cm.

The invention relates to a gel, in particular for use in a wound treatment agent, on mucosa, on regions of the skin which are well suffused with blood, for example also genitals, or other applications for human or animal use.

A method, also referred to as “Aseca method”, for producing an electrochemically activated salt solution, also referred to as anolyte or “Aseca solution”, is described in EP 2 374 762 A1. The disclosure of EP 2 374 762 A1 is expressly included (“incorporation by reference”) with respect to the production method, the medium flow guidance provided for this, the operating parameters provided, the installation used for production and the substance properties named.

The electrochemically activated salt solution (anolyte, “Aseca solution”) produced with the method described in EP 2 374 762 A1 is a highly effective disinfectant with unusual microbiocidal properties which can be obtained in particular by electrolysis of sodium chloride solutions. Such anolyte may for example, depending on the dilution chosen, be used in surface-disinfection applications, e.g. for work surfaces, tables, floors, but also for cold disinfection operations, in agriculture for combating microbial organisms, for washing laundry, in applications for swimming pools or even as prophylaxis against athlete's foot. If required, and in a suitable formulation, such anolyte can however also be used as an active substance for combating microbes or germs of any type, in particular bacteria, viruses, fungi or the like.

Inter alia, it is also desirable to use such an anolyte or such an electrochemically activated salt solution which has inter alia a content of free chlorine of more than 300 mg/l as an agent for the treatment of wounds, in particular for cleaning and disinfecting wounds in order to accelerate the healing process. For this purpose, or alternatively generally in order to extend the possibilities of application, it is desirable to provide the anolyte or the electrochemically activated salt solution in a form which on the one hand permits comparatively simple handling, in particular when applying it to the wounds to be treated, but on the other hand also permits high microbiocidal efficacy of the active substance with concomitant particularly long durability, i.e. in particular high long-term stability. For this purpose, it is attempted to hold the electrochemically activated salt solution, i.e. the anolyte, in the form of a gel, which in addition to the conditions mentioned ensures particularly good storage stability and long-term stability in particular with regard to the microbiocidal efficacy of the anolyte.

The object underlying the invention is therefore to provide a gel, in particular for use in a wound treatment agent or as a wound treatment agent, which meets the aforementioned requirements in a particular way. In addition, a method which is particularly suitable for the production of the gel should be provided.

With respect to the gel, this object is achieved according to the invention in that the gel comprises an inorganic silicate as thickener and also an electrochemically activated aqueous common salt solution, wherein the electrochemically activated aqueous common salt solution has a content of free chlorine of more than 200 mg/l, wherein the electrochemically activated aqueous common salt solution has a conductivity of at most 12 mS/cm.

Advantageous configurations of the invention are the subject of the dependent claims.

As it has surprisingly turned out, among the large number of possible thickeners or gelling agents the desired combination of properties for the gel can be achieved by selecting an inorganic silicate as thickener. By selecting such a material as thickener, in particular the properties of the electrochemically activated common salt solution which are otherwise desired, in particular the microbiocidal efficacy, are retained even after the gel formation process

As it has surprisingly turned out, the electrical conductivity of the electrochemically activated aqueous common salt solution is a particularly significant parameter for keeping to the aforementioned requirements, i.e. retaining the high microbiocidal efficacy of the anolyte or the electrochemically activated common salt solution even over long storage periods, i.e. for high long-term stability. In particular, in this case when producing a gel the desired high long-term stability can be achieved by setting an electrical conductivity of the electrochemically activated aqueous common salt solution of at most 12 mS/cm. In principle, for this purpose the electrochemically activated common salt solution can be produced suitably, i.e. with correspondingly selected conductivity. Alternatively or in a preferred configuration, the electrochemically activated common salt solution may however also be made available by suitable mixing or dilution of an electrochemically activated starting common salt solution (anolyte) with distilled water. The electrochemically activated aqueous common salt solution is thus obtainable by mixing electrochemically activated starting common salt solution which has a content of free chlorine of more than 300 mg/l with distilled water. Particularly preferably, in this case the anolyte or the electrochemically activated salt solution, as is obtainable with the method described in EP 2 374 762 A1, is used as starting common salt solution. Preferably thus “Aseca solution” is used as starting common salt solution.

In order to ensure the desired high microbiocidal efficacy of the gel, the starting common salt solution, i.e. particularly preferably the aforementioned anolyte or in particular the “Aseca solution”, advantageously has a content of free chlorine of more than 500 mg/l, particularly preferably of more than 600 mg/l, and/or a redox potential of between 600 and 100 mV, particularly preferably between 700 and 900 mV, in particular of about 800 mV. In an alternative or additional advantageous configuration, the starting common salt solution, i.e. particularly preferably the anolyte or the “Aseca solution”, has a conductivity of between 12 and 16 mS/cm, particularly advantageously of about 14 mS/cm, and/or a pH value of between 6 and 8, particularly preferably of about 7.

As it has surprisingly turned out, the conductivity of the electrochemically activated aqueous common salt solution obtained by diluting or mixing the electrochemically activated starting common salt solution (in particular anolyte or “Aseca solution”) with distilled water is of particular significance for the thickening or gel formation process, in particular in combination with the intended use of an inorganic silicate as thickener. According to the invention, provision is therefore made to set the electrical conductivity of the aqueous common salt solution obtainable by mixing or diluting the starting common salt solution with distilled water by suitably selecting the mixture ratio or dilution ratio to a value which is particularly suitable for encouraging the thickening or gel formation process. For this, in the preferred case of resorting to anolyte or “Aseca solution”, as is obtainable according to the method described in EP 2 374 762 A1, an electrical conductivity of the diluted—i.e. mixed with distilled water—common salt solution of between 10 and 12 mS/cm, particularly preferably of about 11 mS/cm, is particularly advantageous.

The inorganic silicate intended for use as a thickener or gelling agent, precisely in combination with the particularly preferably selected anolyte (“Aseca solution”), in an advantageous configuration is a lithium magnesium sodium silicate, particularly advantageously from the montmorillonite group, most particularly advantageously Lucentite SWN. The Lucentite SWN which is most particularly preferably provided is obtainable as lithium magnesium sodium silicate, for example under the CAS numbers 53320-86-8, 149316-65-4 or 1217347 or under EINECS No. 258-476-2. Lucentite SWN is a lithium magnesium sodium silicate of the montmorillonite group, and thus a sheet silicate, and corresponds substantially also to Lucentite SAN, but without quaternium-18 being added thereto. This gelling agent is particularly preferred in particular owing to its swelling capacity under low shear forces and in the present case is precisely particularly suitable for a combination with the electrochemically activated common salt solution, since it cannot be oxidised further and thus does not adversely affect the retention of the efficacy and reactivity of the common salt solution. In addition, it is usually produced by synthesis and therefore is particularly efficient owing to its freedom from impurities. Other water-absorbing gelling agents, in particular those from the montmorillonite group, may however likewise be suitable. A more extensive discussion of Lucentite SWN and the related Lucentite SAN can be gathered for example from U.S. Pat. No. 6,660,277 B1 and the U.S. patent application Ser. No. 08/853,992 mentioned therein.

To produce the gel, in particular using the above-mentioned aqueous common salt solution in combination with Lucentite SWN as thickener or gelling agent, the mixture ratio of thickener/common salt solution can be selected suitably corresponding to the desired purpose, i.e. the desired viscosity. For comparatively viscous applications which are not intended to drip, preferably a mixture ratio of thickener/common salt solution of between about 3 and about 5 percent by weight (proportion of thickener), particularly preferably of about 4 percent by weight, is selected. For low-viscosity gels which are intended for example to flow into a wound or the like, preferably a mixture ratio of between 2 and 4 percent by weight (thickener) can be selected. For particularly low-viscosity preferred applications, for example for atomised agents or plant protection products which do not easily drip off, on the other hand, preferably a mixture ratio of between 0.1 and 2 percent by weight (proportion of thickener) is selected. Particularly preferably, the gel is produced using the constituents mentioned such that particular ease of manageability for the intended purpose, i.e. in particular as a wound treatment agent or as a constituent of a wound treatment agent, results. For this, it is advantageously provided, inter alia, to produce the gel such that it has a viscosity of between 4500 and 6500 mPas, particularly preferably of about 5500 mPas.

As it has in addition surprisingly turned out, the gel produced is particularly well suited precisely also for high long-term stability and storage stability while retaining the high microbiocidal efficacy of the starting common salt solution, in particular of the anolyte or the “Areca solution” which is preferably used. Precisely due to the combination of the starting common salt solution with the inorganic silicate selected as thickener, obviously a particularly beneficial incorporation of the active substance in the thickener can take place, so that an exchange of material with the surrounding area, which might encourage the decomposition of the active substance, is prevented in the manner of an encapsulation of the active substance. Thus a particularly beneficial stabilising effect for the active substance can be achieved by the incorporation of the electrochemically activated starting common salt solution (in particular analyte or “Areca solution”) into the gel.

As it has in addition surprisingly turned out, the pH value of the gel produced is of particular significance for the desired high long-term stability and storage stability of the gel. Therefore provision is made to set the pH value of the gel suitably with regard to the constituents used. For the constituents which are particularly preferably provided, namely in particular anolyte or “Areca solution”, and suitable dilution with distilled water to provide the aqueous common salt solution, in conjunction with Lucentite SWN as thickener or gelling agent, a pH value of at least 9, particularly preferably of about 10, is particularly well suited for particularly high long-term stability. Advantageously, when producing the gel, therefore, a pH value of about 10 for the gel is set in particular by suitably selecting the mixture ratio of thickener/common salt solution.

With respect to the method, the named object is achieved in that electrochemically activated starting common salt solution which has a content of chlorine of more than 300 mg/l, with monitoring of the electrical conductivity, is mixed with distilled water by metered addition such that the electrical conductivity of the electrochemically activated aqueous common salt solution produced by the mixing drops to a value of less than 12 mS/cm, preferably to a value of about 11 mS/cm, an inorganic silicate then being admixed as thickener to the aqueous common salt solution.

Preferably in this case a lithium magnesium sodium silicate, preferably a silicate from the montmorillonite group, particularly preferably Lucentite SWN, is admixed as inorganic silicate.

In a particularly advantageous configuration, and in particular in order to ensure the desired high long-term stability and storability of the gel while maintaining a high microbiocidal efficacy, the thickener or the gelling agent in this case is admixed such that the resultant gel has a pH value of at least 9, preferably of between 9 and 10.

The advantages achieved with the invention are in particular that, based on an electrochemically activated starting common salt solution with a content of free chlorine of more than 300 mg/l, by dilution of the starting common salt solution in distilled water such that the resultant aqueous common salt solution has an electrical conductivity of at most 12 mS/cm, and by addition of an inorganic silicate as thickener or gelling agent, a gel with a particularly high microbiocidal action which is suitable for use as a wound treatment agent can be reliably provided. The gel additionally, when being used as a wound treatment agent, also exhibits a cooling action which is perceived to be particularly pleasant. Precisely due to the setting of the pH value of the gel to a value of about 10 which is provided particularly preferably, in particular by suitable selection of the mixture ratios, in this case a particularly high long-term stability and storability of the gel without excessive undesirable loss of microbiocidal efficacy can be achieved. Precisely in the combination with Lucentite SWN, in this case a gel can be provided which while retaining the microbiocidal efficacy is stable in storage and of stable form, and retains its consistency, without tending to become runny or to deliquesce.

An embodiment of the invention, in particular relating to the production of the gel, will be explained in greater detail below.

In the present embodiment the electrochemically activated salt solution which is obtainable by production according to the method described in EP 2 374 762 A1 in the installation disclosed there, and which is referred to as “anolyte” or as “Aseca solution”, is used as base substance or starting common salt solution. Such an anolyte or “Aseca solution” has a content of free chlorine of about 700 mg/I, a redox potential of about 800 mV, a pH value of about 7 and an electrical conductivity of about 14 mS/cm. Such an anolyte or “Aseca solution” is produced according to the method described in EP 2 374 762 A1 electrolytically and by electrochemical activation of a common salt solution. Alternatively, however, a suitable common salt solution which is produced in a different way could of course also be used as starting material.

This electrochemically activated starting common salt solution is diluted by mixing with distilled water and provided with thickener. In principle, in this case initially a preliminary product consisting of thickener and distilled water could be produced, into which the electrochemically activated starting common salt solution, i.e. in the present case anolyte or “Aseca solution”, is then incorporated. It is however preferred, as described below, for the electrochemically activated starting common salt solution first to be diluted with distilled water and only then for the thickener or gelling agent to be added.

For this, the electrochemically activated starting common salt solution, i.e. anolyte or “Aseca solution”, is diluted by admixing distilled water. The dilution in this case takes place by metered addition of distilled water, with the electrical conductivity of the diluted, aqueous common salt solution being measured as a suitable parameter for process management. The addition of distilled water, i.e. the dilution, in this case is carried out in such a manner and for such a time until the conductivity of the mixture, i.e. of the dilute, aqueous common salt solution, has dropped to a value of about 11 mS/cm.

Then Lucentite SWN, i.e. an inorganic silicate, is admixed as thickener or gelling agent to the aqueous common salt solution which is thus prepared. The addition of the thickener in this case takes place approximately in a mixture ratio of thickener/common salt solution of about 4%, with inter alia the viscosity of the resultant gel being determined as a parameter of the process management. This is advantageously set to a target value of about 5500 mPa. Furthermore, the pH value of the gel is determined as a parameter which is significant precisely for the long-term stability and storability of the resultant gel. The thickener is in this case fed in such that the resultant gel has a pH value of at least 9, preferably of about 10.

The addition of the thickener or gelling agent in this case takes place by successive scattering into the mixture with stirring. Then first of all, with further stirring, initial swelling takes place, and then, with an increase in stirring speed, homogenisation.

1.-13. (canceled)

14. A gel, comprising:
an inorganic silicate as thickener; and
an electrochemically activated aqueous common salt solution having a content of free chlorine of more than 200 mg/l and a conductivity of not more than 12 mS/cm.

15. The gel of claim 14, for use in a wound treatment agent.

16. The gel of claim 14, wherein the electrochemically activated aqueous common salt solution is obtained by mixing an electrochemically activated starting common salt solution having a content of free chlorine of more than 300 mg/l with distilled water.

17. The gel of claim 14, wherein a mixture ratio of thickener/common salt solution is between about 3 and about 5% by weight.

18. The gel of claim 16, wherein the starting common salt solution has a content of free chlorine of more than 500 mg/l, preferably of more than 600 mg/l.

19. The gel of claim 16, wherein the starting common salt solution has a redox potential of between 600 and 1000 mV, preferably between 700 and 900 mV, particularly preferably of about 800 mV.

20. The gel of claim 16, wherein the starting common salt solution has a conductivity of between 12 and 16 mS/cm.

21. The gel of claim 16, wherein the starting common salt solution has a pH value of between 6 and 8, preferably of about 7.

22. The gel of claim 14, wherein the common salt solution has a conductivity of between 10 and 12 mS/cm, preferably of about 11 mS/cm.

23. The gel of claim 14, wherein the thickener is a lithium-magnesium-sodium silicate.

24. The gel of claim 14, wherein the thickener is a silicate from the montmorillonite group.

25. The gel of claim 14, wherein the thickener is Lucentite SWN.

26. The gel of claim 14, having a pH value of at least 9, preferably of between 9 and 10, and/or a viscosity of between 4500 and 6500 mPa, preferably about 5500 mPa.

27. A method for producing a gel for use in a wound treatment agent, comprising:
providing an electrochemically activated starting common salt solution having a content of free chlorine of more than 300 mg/l;
producing an electrochemically activated aqueous common salt solution having an electrical conductivity of less than 12 mS/cm, preferably of 11 mS/cm by mixing the electrochemically activated starting common salt solution with distilled water by metered addition of the distilled water to the electrochemically activated starting common salt solution; and
admixing an inorganic silicate as thickener to the electrochemically activated aqueous common salt solution.

28. The method of claim 27, wherein the inorganic silicate is a lithium-magnesium-silicate,

29. The method of claim 27, wherein the inorganic silicate is a silicate from the montmorillonite group.

30. The method of claim 27, wherein the inorganic silicate is Lucenite SWN.

31. The method of claim 27, wherein the thickener is admixed such that the resultant gel has a pH value of at least 9, preferably of between 9 and 10.
